(19) United States
(12) Patent Application Publication
(10) Pub. No.: US 2025/0291528 A1
(43) Pub. Date: Sep. 18, 2025

(54) INFORMATION MANAGEMENT SYSTEM, MANAGEMENT DEVICE, AND NON-TRANSITORY COMPUTER-READABLE STORAGE MEDIUM STORING PROGRAM

(71) Applicant: SEIKO EPSON CORPORATION, Tokyo (JP)

(72) Inventors: Yusuke NAKANO, MATSUMOTO-SHI (JP); Hiroyuki KURAMOTO, SHIOJIRI-SHI (JP)

(21) Appl. No.: 19/081,605

(22) Filed: Mar. 17, 2025

(30) Foreign Application Priority Data
Mar. 18, 2024 (JP) 2024-042101

(51) Int. Cl. G06F 3/12

(52) U.S. Cl. CPC G06F 3/1234; G06F 3/121; G06F 3/1273

(57) ABSTRACT

There is provided an information management system including a management system configured to manage management information, a print system configured to cause a printing apparatus to print the management information, and a management device configured to acquire the management information from scan data of a first print on which the management information is printed and store the acquired management information in a database on an isolated network isolated from an external network. The print system causes the printing apparatus to print a print history of the management information, and the management device acquires the print history of the management information from scan data of a second print on which a print history of the management information is printed and notifies storage omission of the management information in the database based on the acquired print history of the management information and a scan history of the first print.

The present application is based on, and claims priority from JP Application Serial Number 2024-042101, filed Mar. 18, 2024, the disclosure of which is hereby incorporated by reference herein in its entirety.

### BACKGROUND

### 1. Technical Field

The present disclosure relates to an information management system, a management device, and a non-transitory computer-readable storage medium storing a program.

### 2. Related Art

There has been known a technique for managing information on a network. For example, JP-A-2022-063904 discloses a system in which a cloud server on a cloud network manages medical information such as an electronic medical record.

JP-A-2022-063904 is an example of the related art.

The management information such as the medical information described in JP-A-2022-063904 is sometimes managed by a database on an isolated network. In this case, as a configuration in which the management information is stored in the database, a configuration is conceivable in which a print on which the management information is printed is read by a scanner and the management information obtained from scan data is stored in the database. However, in this configuration, storage omission of the management information in the database can occur because of a loss of the print, double feeding occurring in the scanner, or the like.

### SUMMARY

According to an aspect of the present disclosure, there is provided an information management system including: a management system configured to manage management information; a print system configured to cause a printing apparatus to print the management information acquired from the management system; and a management device configured to acquire the management information from scan data of a first print on which the management information is printed and store the acquired management information in a database on an isolated network isolated from an external network, wherein the print system causes the printing apparatus to print a print history of the management information, and the management device acquires the print history of the management information from scan data of a second print on which a print history of the management information is printed and notifies storage omission of the management information in the database based on the acquired print history of the management information and a scan history of the first print.

According to another aspect of the present disclosure, there is provided a management device connected to an isolated network isolated from an external network to which a management system that manages management information and a print system that causes a printing apparatus to print the management information acquired from the management system are connected, the management device including a management device control unit configured to acquire the management information from scan data of a first print on which the management information is printed and store the acquired management information in a database on the isolated network, wherein the management device control unit acquires a print history of the management information from scan data of a print that the print system caused the printing apparatus to print, the print being a second print on which the print history of the management information is printed, and notifies storage omission of the management information in the database based on the acquired print history of the management information and a scan history of the first print.

According to still another aspect of the present disclosure, there is provided a non-transitory computer-readable storage medium storing a program, the program causing a processor of a management device connected to an isolated network separated from an external network, to which a management system that manages management information and a print system that causes a printing apparatus to print the management information acquired from the management system are connected, to acquire the management information from scan data of a first print on which the management information is printed; store the acquired management information in a database on the isolated network; acquire a print history of the management information from scan data of a print that the print system caused the printing apparatus to print, the print being a second print on which the print history of the management information is printed; and notify storage omission of the management information in the database based on the acquired print history of the management information and a scan history of the first print.

According to still another aspect of the present disclosure, there is provided a server connected to, via an external network, a management system that manages management information, the server causing a printing apparatus to print the management information acquired from the management system, the server including a server control unit configured to cause the printing apparatus to generate a first print on which the management information is printed and cause the printing apparatus to generate a second print on which a print history of the management information is printed, wherein the first print is a print that is a target of scan data from which a management device, which stores the management information in a database on an isolated network isolated from the external network, acquires the management information, and the second print is a print on which a print history of the management information to be compared with a scan history of the first print is printed when storage omission of the management information in the database is notified.

### BRIEF DESCRIPTION OF THE DRAWINGS

FIG. 1 is a diagram illustrating a configuration of an information management system.

FIG. 2 is a diagram illustrating configurations of an inquiry management server and a print server.

FIG. 3 is a diagram illustrating an example of a first print image.

FIG. 4 is a diagram illustrating an example of a second print image.

FIG. 5 is a diagram illustrating a configuration of a second in-hospital terminal.

FIG. 6 is a flowchart illustrating operations of units of the information management system.

FIG. 7 is a flowchart illustrating operations of the units of the information management system.

FIG. 8 is a flowchart illustrating operations of the units of the information management system.

FIG. 9 is a flowchart illustrating operations of the units of the information management system.

FIG. 10 is a diagram illustrating an example of a notification screen.

FIG. 11 is a diagram illustrating configurations of the inquiry management server and the print server.

FIG. 12 is a flowchart illustrating operations of the units of the information management system.

FIG. 13 is a flowchart illustrating operations of the units of the information management system.

### DESCRIPTION OF EMBODIMENTS

### 1. First Embodiment

### 1-1. Configuration of an Information Management System

First, a first embodiment is explained.

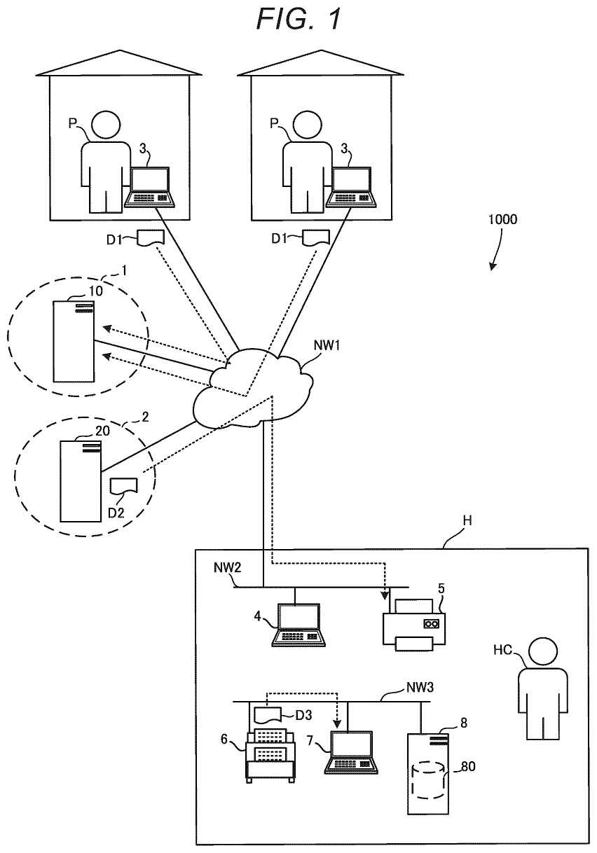

FIG. 1 is a diagram illustrating a configuration of an information management system 1000.

The information management system 1000 according to the present embodiment is a system applied to, for example, a hospital H and is a system that manages inquiry answer information J1. The inquiry answer information J1 is information answered by a patient P to an inquiry. The inquiry answer information J1 includes, for example, a drug history answered by the patient P, a medical history answered by the patient P, and a medical examination history answered by the patient P.

The inquiry answer information J1 is an example of “management information”.

The hospital H in the present embodiment is an institution that provides medical treatment to the patient P and is not limited to a facility that accommodates the patient P. In other words, the hospital H in the present embodiment may be an institution that provides medical treatment to an outpatient or may be an institution that provides medical treatment to a patient with an outpatient and an inpatient.

The information management system 1000 includes an inquiry management system 1 connected to an external network NW1 that is a Wide Area Network (WAN) such as the Internet.

The inquiry management system 1 is a system that manages the inquiry answer information J1. The inquiry management system 1 includes an inquiry management server 10. The inquiry management server 10 is a server device that manages the inquiry answer information J1 and is a server device that processes information with a print system 2, a patient terminal 3 used by the patient P, and a first in-hospital terminal 4 used in the hospital H as clients. The inquiry management server 10 is connected to an external network NW1 and communicates with these kinds of equipment. In the drawings, the inquiry management server 10 is represented by one block. However, this does not always mean that the inquiry management server 10 is configured by a single device. For example, the inquiry management server 10 may include a plurality of server devices having different processing contents.

The inquiry management system 1 is an example of a “management system”.

The patient terminal 3 is a terminal device used by the patient P. FIG. 1 exemplifies a case in which the patient terminal 3 is a laptop PC. However, the patient terminal 3 may be a desktop PC, a tablet PC, or a smartphone. The patient terminal 3 is connected to the external network NW1 and communicates with the inquiry management system 1. The patient terminal 3 displays an input screen for an inquiry at a patient home or the like and receives input of an answer to the inquiry from the patient P. When the patient P ends the answer to the inquiry, the patient terminal 3 transmits inquiry answer information data D1 indicating the inquiry answer information J1 to the inquiry management system 1.

The information management system 1000 includes the print system 2 connected to the external network NW1.

The print system 2 is a system that causes a printing apparatus 5 used in the hospital H to perform printing. The print system 2 includes a print server 20. The print server 20 is a server device that processes information with the inquiry management system 1 and the printing apparatus 5 as clients. The print server 20 is connected to the external network NW1 and communicates with these kinds of equipment. In the drawings, the print server 20 is represented by one block. However, this does not always mean that the print server 20 is configured by a single device. For example, the print server 20 may include a plurality of server devices having different processing contents.

The print server 20 is an example of a “server”.

The information management system 1000 includes the printing apparatus 5.

The printing apparatus 5 is a device that performs printing using a print head to generate a print. The printing apparatus 5 is installed in the hospital H and connected to an external connection network NW2 constructed in the hospital H. The external connection network NW2 is a network connected to the external network NW1. For this reason, the printing apparatus 5 is capable of communicating with the first in-hospital terminal 4 connected to the external connection network NW2 and the print system 2 connected to the external network NW1. The printing apparatus 5 in the present embodiment receives print data D2 from the print system 2 and performs printing based on the received print data D2. The print data D2 is data including image data of an image formed on a print and a control command conforming to a command system of the printing apparatus 5.

The printing apparatus 5 may be an inkjet type printing apparatus or may be an electronic transfer type printing apparatus. When the printing apparatus 5 is the inkjet type, a format of a printing head provided in the printing apparatus 5 may be a serial type, may be a line type, or may be a lateral type. Besides a print function, the printing apparatus 5 may have functions such as a scan function and a facsimile function.

As illustrated in FIG. 1, besides the printing apparatus 5, the first in-hospital terminal 4 is connected to the external connection network NW2.

The first in-hospital terminal 4 is a terminal device used by a medical worker HC. The medical worker HC includes a doctor, a nurse, a person engaged in office work relating to the hospital H, and the like. FIG. 1 exemplifies a case in which the first in-hospital terminal 4 is a laptop PC. However, the first in-hospital terminal 4 may be a desktop PC or may be a tablet PC. The first in-hospital terminal 4 is connected to the external connection network NW2 and communicates with the inquiry management system 1 and the printing apparatus 5.

The information management system 1000 includes a scanner 6.

The scanner 6 is a device that performs scanning. The scanner 6 is installed in the hospital H and connected to an isolated network NW3 constructed in the hospital H. The isolated network NW3 is a network isolated from the external network NW1. That is, the isolated network NW3 is not connected to the external network NW1. The scanner 6 transmits scan data D3 obtained by the scanning to a second in-hospital terminal 7 connected to the isolated network NW3.

The second in-hospital terminal 7 is an example of a “management device”.

The information management system 1000 includes the second in-hospital terminal 7.

The second in-hospital terminal 7 is a terminal device installed in the hospital H and used by the medical worker HC. FIG. 1 exemplifies a case in which the second in-hospital terminal 7 is a laptop PC. However, the second in-hospital terminal 7 may be a desktop PC or may be a tablet PC. The second in-hospital terminal 7 is connected to the isolated network NW3 and communicates with the scanner 6 and the storage device 8.

The information management system 1000 includes a storage device 8.

The storage device 8 is a device that includes a database 80 and stores the inquiry answer information J1 in the database 80 as an electronic medical record. The storage device 8 is connected to the isolated network NW3 and communicates with the second in-hospital terminal 7.

### 1-2. Configuration of the Server

Subsequently, configurations of the inquiry management server 10 and the print server 20 are explained below.

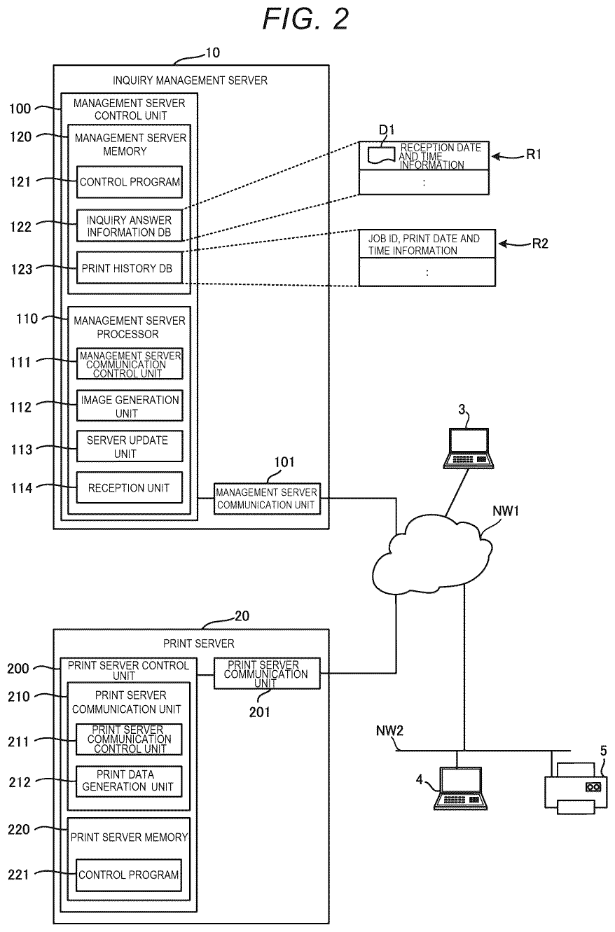

FIG. 2 is a diagram illustrating the configurations of the inquiry management server 10 and the print server 20.

First, the configuration of the inquiry management server 10 is explained.

The inquiry management server 10 includes a management server control unit 100 and a management server communication unit 101.

The management server communication unit 101 includes communication hardware conforming to a predetermined communication standard such as a communication circuit and communicates with the print system 2, the patient terminal 3, and the first in-hospital terminal 4 according to control of the management server control unit 100.

The management server control unit 100 includes a management server processor 110 such as a central processing unit (CPU) or a micro processing unit (MPU) and a management server memory 120. The management server communication unit 101 is coupled to the management server control unit 100.

The management server memory 120 is a memory that stores programs and data. The management server memory 120 stores a control program 121, an inquiry answer information DB (database) 122, a print history DB 123, data to be processed by the management server processor 110, and the like. The management server memory 120 includes a nonvolatile storage area. The management server memory 120 includes a volatile storage area and constitutes a work area of the management server processor 110. The management server memory 120 is configured by, for example, a ROM (Read Only Memory) or a RAM (Random Access Memory).

The inquiry answer information DB 122 is a database having a record R1 for each piece of the inquiry answer information J1. The record R1 includes inquiry answer information data D1 and consultation date and time information indicating a date and time when the inquiry answer information data D1 is received from the patient terminal 3.

The print history DB 123 is a database having records R2 for each piece of the inquiry answer information J1 on which printing has been performed. The record R2 includes a job ID (Identification) for identifying a first print on which the inquiry answer information J1 is printed and print date and time information indicating a print date and time of the inquiry answer information J1.

The job ID is an example of “identification information”.

The management server processor 110 reads and executes the control program 121 stored by the management server memory 120 to function as a management server communication control unit 111, an image generation unit 112, a server update unit 113, and a reception unit 114.

The management server communication control unit 111 communicates with the print system 2, the patient terminal 3, and the first in-hospital terminal 4 via the management server communication unit 101.

The image generation unit 112 generates a print image to be printed by the printing apparatus 5.

When the management server communication control unit 111 receives the inquiry answer information data D1 from the patient terminal 3, the image generation unit 112 generates a first print image PG1 based on the inquiry answer information data D1 received by the management server communication control unit 111.

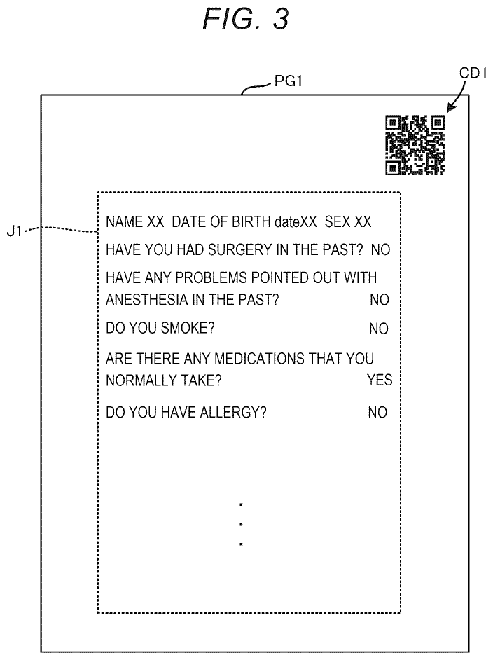

FIG. 3 is a diagram illustrating an example of the first print image PG1.

The first print image PG1 includes the inquiry answer information J1.

The first print image PG1 includes a first code CD1. In FIG. 3, a QR code (registered trademark), which is a two-dimensional code, is exemplified as the first code CD1. However, the first code CD1 included in the first print image PG1 may be a one-dimensional code.

The first code CD1 is an example of a “code”.

When generating the first print image PG1, the image generation unit 112 generates a job ID. Subsequently, the image generation unit 112 generates the first code CD1 in which the generated job ID and the inquiry answer information J1 indicated by the inquiry answer information data D1 are recorded. Then, the image generation unit 112 generates the first print image PG1 including the generated first code CD1 and the inquiry answer information J1 indicated by the inquiry answer information data D1.

After generating the first print image PG1, the image generation unit 112 outputs image data of the generated first print image PG1 to the management server communication control unit 111. When receiving the image data of the first print image PG1, the management server communication control unit 111 transmits the received image data of the first print image PG1 to the print system 2 together with information designating the printing apparatus 5 at a printing destination.

The image generation unit 112 generates a second print image PG2 referring to the print history DB 123.

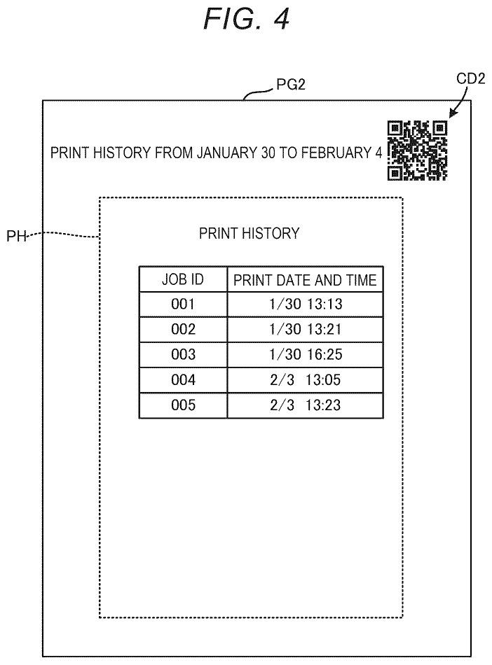

FIG. 4 is a diagram illustrating an example of the second print image PG2.

The second print image PG2 includes a print history PH of the inquiry answer information J1. The print history PH included in the second print image PG2 is a history of the inquiry answer information J1 printed before the second print image PG2 is generated this time after the second print image PG2 is generated last time. The print history PH is history information including at least a job ID of a printed job. In the present embodiment, it is assumed that the print history PH is history information including a combination of a job ID and a print date and time for each piece of the printed inquiry answer information J1.

The second print image PG2 includes a second code CD2. In FIG. 4, a QR code, which is a two-dimensional code, is exemplified as the second code CD2. However, the second code CD2 included in the second print image PG2 may be a one-dimensional code.

The image generation unit 112 refers to the print history DB 123 and specifies the record R2 of the inquiry answer information J1 printed before the second print image PG2 is generated this time after the second print image PG2 is generated last time. For example, in records R2 of the print history DB 123, a flag indicating whether the record R2 has been specified is added to the image generation unit 112. The image generation unit 112 specifies, based on a value of the flag, the record R2 of the inquiry answer information J1 printed before the second print image PG2 is generated this time after the second print image PG2 is generated last time.

Subsequently, the image generation unit 112 reads a job ID and print date and time information for each specified record R2. Subsequently, the image generation unit 112 generates the print history PH indicating a list of combinations of the read job ID and a print date and time indicated by the read print date and time information. The image generation unit 112 generates the second code CD2 in which the generated print history PH is recorded. Then, the image generation unit 112 generates the second print image PG2 including the generated print history PH and the generated second code CD2.

After the second print image PG2 is generated, the image generation unit 112 outputs image data of the generated second print image PG2 to the management server communication control unit 111. When receiving the image data of the second print image PG2, the management server communication control unit 111 transmits the received image data of the second print image PG2 to the print system 2 together with information designating the printing apparatus 5 at a printing destination.

The server update unit 113 updates the inquiry answer information DB 122. When the management server communication control unit 111 receives the inquiry answer information data D1 from the patient terminal 3, the server update unit 113 generates the record R1 including the inquiry answer information data D1 and stores the generated record R1 in the inquiry answer information DB 122.

The server update unit 113 updates the print history DB 123. When the management server communication control unit 111 transmits the image data of the first print image PG1 to the print system 2, the server update unit 113 generates the record R2 and stores the generated record R2 in the print history DB 123. When generating the record R2, the server update unit 113 acquires the job ID generated at the time of generating the first print image PG1 from the image generation unit 112. Then, the server update unit 113 generates the record R2 including the acquired job ID and print date and time information. A print date and time indicated by the print date and time information included in the record R2 may be a date and time when the image data of the first print image PG1 was transmitted to the print system 2, may be a date and time when the print system 2 caused the printing apparatus 5 to print the first print image PG1, or may be a date and time when the printing apparatus 5 completed the printing of the first print image PG1.

The reception unit 114 receives, from the first in-hospital terminal 4, designation of print time when the print history PH is printed. The first in-hospital terminal 4 receives designation of desired print time from the medical worker HC and notifies the print time designated by the medical worker HC to the inquiry management system 1. When the management server communication control unit 111 receives the notification, the reception unit 114 receives the print time designated by the medical worker HC.

Subsequently, the configuration of the print server 20 is explained.

The print server 20 includes a print server control unit 200 and a print server communication unit 201.

The print server control unit 200 is an example of a “server control unit”.

The print server communication unit 201 includes communication hardware conforming to a predetermined communication standard such as a communication circuit and communicates with the inquiry management system 1 and the printing apparatus 5 according to the control of the print server control unit 200.

The print server control unit 200 includes a print server processor 210 such as a central processing unit (CPU) or a micro-processing unit (MPU) and a print server memory 220. The print server communication unit 201 is coupled to the print server control unit 200.

The print server memory 220 is a memory that stores programs and data. The print server memory 220 stores a control program 221 and data to be processed by the print server processor 210. The print server memory 220 has a nonvolatile storage area. The print server memory 220 includes a volatile storage area and configures a work area of the print server processor 210. The print server memory 220 is configured by, for example, a read only memory (ROM) or a random access memory (RAM).

The print server processor 210 reads and executes the control program 221 stored by the print server memory 220 to function as a print server communication control unit 211 and a print data generation unit 212.

The print server communication control unit 211 communicates with the inquiry management system 1 and the printing apparatus 5 via the print server communication unit 201.

The print data generation unit 212 generates the print data D2 based on image data received from the inquiry management system 1. When after generating the print data D2, the print data generation unit 212 outputs the generated print data D2 to the print server communication control unit 211. When receiving the print data D2, the print server communication control unit 211 transmits the received print data D2 to the printing apparatus 5 designated from the inquiry management system 1. Accordingly, for example, when the print server 20 of the print system 2 receives image data of the first print image PG1 from the information management system 1000, the print server 20 causes the printing apparatus 5 to execute printing of the first print image PG1 and a first print corresponding to the first print image PG1 is obtained. On the other hand, when image data of the second print image PG2 is received from the information management system 1000, the print server 20 causes the printing apparatus 5 to print the second print image PG2 and a second print corresponding to the second print image PG2 is obtained.

1-3. Configuration of the Second in-Hospital Terminal

Next, a configuration of the second in-hospital terminal 7 is explained.

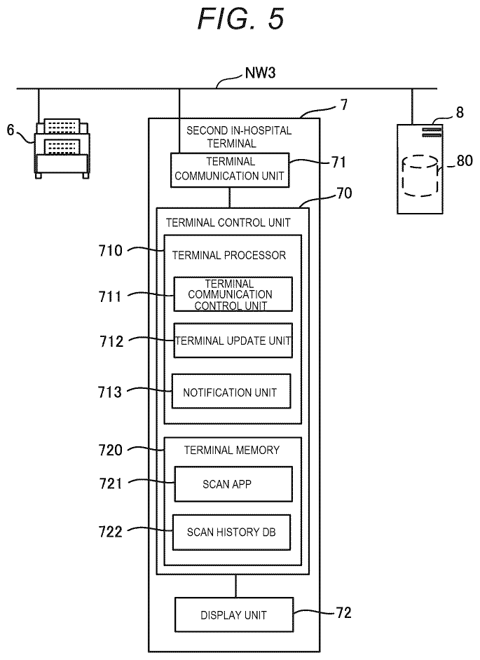

FIG. 5 is a diagram illustrating the configuration of the second in-hospital terminal 7.

The second in-hospital terminal 7 includes a terminal control unit 70, a terminal communication unit 71, and a display unit 72.

The terminal control unit 70 is an example of a “management device control unit”.

The terminal communication unit 71 includes communication hardware conforming to a predetermined communication standard such as a communication circuit and communicates with the scanner 6 and the storage device 8 according to control of the terminal control unit 70.

The display unit 72 includes a display and displays information on the display according to the control of the terminal control unit 70.

The terminal control unit 70 includes a terminal processor 710 such as a central processing unit (CPU) or a micro-processing unit (MPU) and a terminal memory 720. The terminal communication unit 71 and the display unit 72 are coupled to the terminal control unit 70.

The terminal processor 710 is an example of a “processor”.

The terminal memory 720 is a memory that stores programs and data. The terminal memory 720 stores a scan application program (hereinafter referred to as “scan APP”) 721, a scan history DB 722, data to be processed by the terminal processor 710, and the like. The terminal memory 720 has a nonvolatile storage area. The terminal memory 720 includes a volatile storage area and configures a work area of the terminal processor 710. The terminal memory 720 is configured by, for example, a read only memory (ROM) or a random access memory (RAM).

The scan APP 721 is an example of a “program”.

The scan APP 721 is an application program for controlling scanning by the scanner 6 and performs processing of generating scan data D3 of an image read by the scanner 6 and processing of storing scan data in a desired storage place.

The scan history DB 722 is a database that manages a scan history. A job ID acquired from the scan data D3 is stored in the scan history DB 722.

The terminal processor 710 reads and executes the scan APP 721 stored in the terminal memory 720 to function as a terminal communication control unit 711, a terminal update unit 712, and a notification unit 713 by.

The terminal communication control unit 711 communicates with the scanner 6 and the storage device 8 via the terminal communication unit 71.

The terminal update unit 712 updates the scan history DB 722. When the terminal communication unit 71 receives the scan data D3 of the first print, the terminal update unit 712 acquires a job ID from a scan image indicated by the scan data D3 of the first print. The scan image indicated by the scan data D3 of the first print includes the first print image PG1 printed on the first print. For that reason, the terminal update unit 712 acquires the job ID by reading the job ID from the first code CD1 of the first print image PG1 included in the scan image. After acquiring the job ID from the scan data D3 of the first print, the terminal update unit 712 stores the acquired job ID in the scan history DB 722.

The notification unit 713 displays information with the display unit 72 to notify the information to the medical worker HC.

### 1-4. Operation Until the Inquiry Answer Information is Stored

Subsequently, operations of the units of the information management system 1000 until the inquiry answer information J1 is stored in the database 80 on the isolated network NW3 is explained.

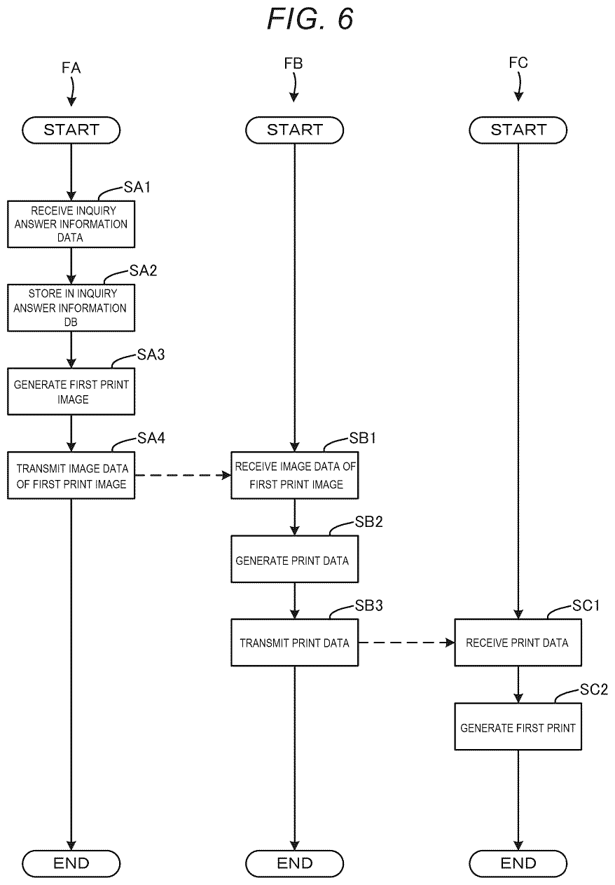

FIG. 6 is a flowchart illustrating the operations of the units of the information management system 1000. In FIG. 6, a flowchart FA illustrates an operation of the inquiry management system 1, a flowchart FB illustrates an operation of the print system 2, and a flowchart FC illustrates an operation of the printing apparatus 5.

When the patient terminal 3 transmits the inquiry answer information data D1 to the inquiry management system 1, the management server communication control unit 111 receives the inquiry answer information data D1 from the patient terminal 3 (step SA1).

Subsequently, the server update unit 113 stores the record R1 including the inquiry answer information data D1 received in step SA1 in the inquiry answer information DB 122 (step SA2).

Subsequently, the image generation unit 112 generates the first print image PG1 based on the inquiry answer information data D1 received in step SA1 (step SA3).

Subsequently, the management server communication control unit 111 transmits the image data of the first print image PG1 generated in step SA3 to the print system 2 (step SA4).

As illustrated in the flowchart FB, the print server communication control unit 211 receives the image data of the first print image PG1 from the inquiry management system 1 (step SB1).

Subsequently, the print data generation unit 212 generates, based on the image data received in step SB1, the print data D2 for printing the first print image PG1 indicated by the image data received in step SB1 (step SB2).

Subsequently, the print server communication control unit 211 transmits the print data D2 generated in step SB2 to the printing apparatus 5 (step SB3).

As illustrated in the flowchart FC, when receiving the print data D2 from the print system 2 (step SC1), the printing apparatus 5 executes printing based on the received print data D2 and generates the first print on which the first print image PG1 generated in step SA3 is printed (step SC2).

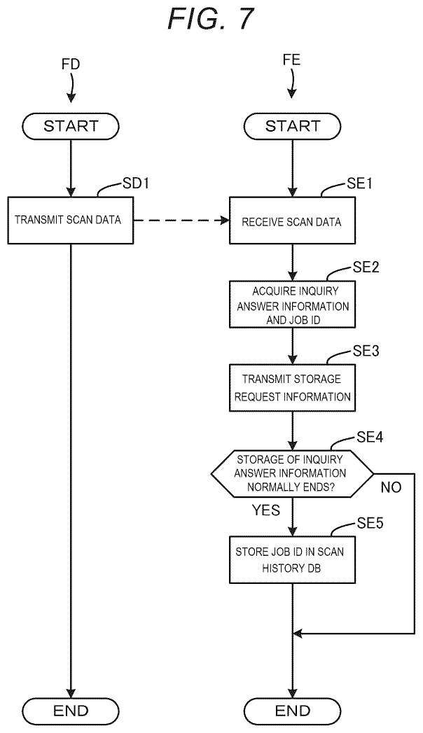

After the first print is generated, the medical worker HC sets generated one or a plurality of first prints on the scanner 6 and performs, on the scanner 6 or a scan application, operation of causing the scanner 6 to scan the first print. Subsequently, an operation of the information management system 1000 in the cases in which the scanner 6 scans the first print is explained with reference to FIG. 7.

FIG. 7 is a flowchart illustrating operations of the units of the information management system 1000. In FIG. 7, a flowchart FD illustrates an operation of the scanner 6 and a flowchart FE illustrates an operation of the second in-hospital terminal 7.

After performing scanning, the scanner 6 transmits the scan data D3 to the second in-hospital terminal 7 (step SD1).

As illustrated in the flowchart FE, the terminal communication control unit 711 of the second in-hospital terminal 7 receives the scan data D3 (step SE1).

Subsequently, the terminal communication control unit 711 acquires the inquiry answer information J1 and the job ID from the first code CD1 included in the scan image indicated by the scan data D3 received in step SE1 (step SE2).

Subsequently, the terminal communication control unit 711 transmits storage request information to the storage device 8 (step SE3). The storage request information is information for requesting storage of the inquiry answer information J1 acquired in step SE2 in the database 80. The storage request information includes the inquiry answer information data D1 indicating the inquiry answer information J1 acquired in step SE2.

When receiving the storage request information, the storage device 8 stores the inquiry answer information J1 in the database 80 by causing the database 80 to store the storage request inquiry answer information in the inquiry answer information data D1. When the storage of the inquiry answer information J1 ends, the storage device 8 returns the normal end of the storage as a response to the storage request information. When the storage of the inquiry answer information J1 does not normally end, the storage device 8 returns the not-normal end of the storage as a response to the storage request information.

The terminal update unit 712 determines whether the storage of the inquiry answer information J1 has normally ended (step SE4). When receiving, from the storage device 8, the response indicating the storage has normally ended, the terminal update unit 712 makes positive determination in step SE4. When receiving, from the storage device 8, the response indicating that the storage has not normally ended, the terminal update unit 712 makes negative determination in step SE4.

When determining that the storage of the inquiry answer information J1 has not normally ended (step SE4: NO), the terminal update unit 712 ends this processing. When the terminal update unit 712 makes the negative determination in step SA4, the notification unit 713 may notify that the storage of the inquiry answer information J1 has not normally ended.

On the other hand, when determining that the storage of the inquiry answer information J1 has normally ended (step SE4: YES), the terminal update unit 712 acquires a job ID from the scan data D3 received in step SE1 and stores the acquired job ID in the scan history DB 722 (step SE5). In the present embodiment, the job ID of the inquiry answer information J1 for which the reading has been performed by the scanning and the storage in the database 80 has normally ended is stored in the scan history DB 722 as the scan history. However, a job ID of the inquiry answer information J1 for which the reading by the scanning has been simply performed may be stored in the scan history DB 722 as the scan history.

### 1-5. Operation Relating to Storage Omission of the Inquiry Answer Information

Subsequently, operations of the units of the information management system 1000 related to storage omission of the inquiry answer information J1 in the database 80 are explained.

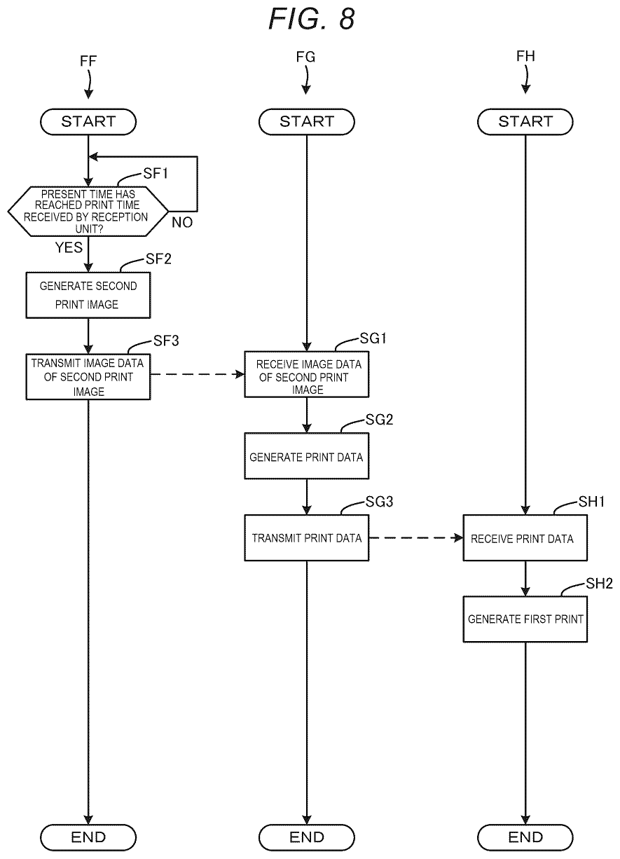

FIG. 8 is a flowchart illustrating the operations of the units of the information management system 1000. In FIG. 8, a flowchart FF illustrates an operation of the inquiry management system 1, a flowchart FG illustrates an operation of the print system 2, and a flowchart FH illustrates an operation of the printing apparatus 5.

The image generation unit 112 determines whether the present time has reached print time received by the reception unit 114 (step SF1). When determining that the present time has not reached the print time received by the reception unit 114 (step SF1: NO), the image generation unit 112 performs the determination in step SF1 again.

When determining that the present time has reached the print time received by the reception unit 114 (step SF1: YES), the image generation unit 112 generates the second print image PG2 (step SF2).

Subsequently, the management server communication control unit 111 transmits image data of the second print image PG2 generated in step SF3 to the print system 2 (step SF3).

As illustrated in the flowchart FG, the print server communication control unit 211 receives the image data of the second print image PG2 from the inquiry management system 1 (step SG1).

Subsequently, the print data generation unit 212 generates, based on the image data received in step SG1, the print data D2 for printing the second print image PG2 indicated by the image data received in step SG1 (step SG2).

Subsequently, the print server communication control unit 211 transmits the print data D2 generated in step SG2 to the printing apparatus 5 (step SG3).

As illustrated in the flowchart FH, when receiving the print data D2 from the print system 2 (step SH1), the printing apparatus 5 executes printing based on the received print data D2 and generates the second print on which the second print image PG2 generated in step SF2 is printed (step SH2).

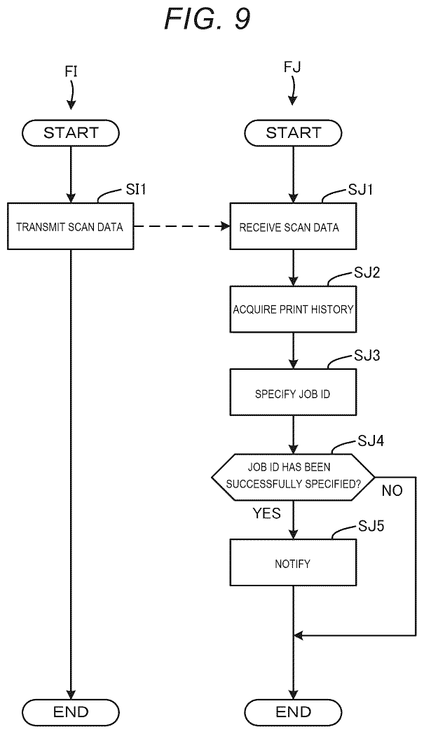

When the second print is generated, the medical worker HC sets the second print on the scanner 6 and performs operation of causing the scanner 6 to scan the second print. Subsequently, an operation of the information management system 1000 in the case in which the scanner 6 scans the second print is explained with reference to FIG. 9.

FIG. 9 is a flowchart illustrating operations of the units of the information management system 1000. In FIG. 9, a flowchart FI indicates an operation of the scanner 6 and the flowchart FJ indicates an operation of the second in-hospital terminal 7.

After performing scanning, the scanner 6 transmits the scan data D3 of the second print to the second in-hospital terminal 7 (step SJ1).

As illustrated in the flowchart FJ, the terminal communication control unit 711 of the second in-hospital terminal 7 receives the scan data D3 (step SJ1).

Subsequently, the notification unit 713 acquires the print history PH from the second code CD2 included in a scan image indicated by the scan data D3 received in step SJ1 (step SJ2).

Subsequently, based on the print history PH acquired in step SJ2 and the scan history indicated by the scan history DB 722, the notification unit 713 specifies a job ID corresponding to the inquiry answer information J1, storage of which in the database 80 is omitted (step SJ3).

The step SJ3 is explained in detail.

For each of job IDs included in the print history PH acquired in step SJ2, the notification unit 713 determines whether the job ID is stored in the scan history DB 722. Then, the notification unit 713 specifies the job ID determined as not being stored in the scan history DB 722 as the job ID corresponding to the inquiry answer information J1, storage of which in the database 80 is omitted.

The notification unit 713 determines whether the job ID corresponding to the inquiry answer information J1, storage of which in the database 80 is omitted, has been specified in step SJ3 (step SJ4).

When determining that the job ID corresponding to the inquiry answer information J1, storage of which in the database 80 is omitted, has not be successfully specified (step SJ4: NO), the notification unit 713 ends this processing.

On the other hand, when determining that the job ID corresponding to the inquiry answer information J1, storage of which in the database 80 is omitted, has been successfully specified (step SJ4: YES), the notification unit 713 notifies the storage omission in the database 80 (step SJ5).

In step SJ5, the notification unit 713 displays a notification screen TG with the display unit 72 to notify the storage omission in the database 80.

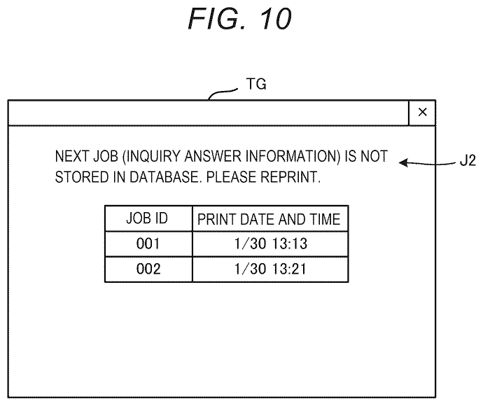

FIG. 10 is a diagram illustrating an example of the notification screen TG.

The notification screen TG is a screen for displaying, as a list, a combination of the job ID corresponding to the inquiry answer information J1, storage of which in the database 80 is omitted, and a print date and time of the inquiry answer information J1 corresponding to the job ID.

The notification screen TG is a screen for displaying reprint information J2 for urging reprinting of the inquiry answer information J1, storage of which in the database 80 is omitted.

The notification unit 713 extracts, from the print history PH acquired in step SJ2, the job ID corresponding to the inquiry answer information J1, storage of which in the database 80 is omitted, and print date and time information associated with the job ID. Subsequently, the notification unit 713 generates the notification screen TG for displaying, as a list, the extracted job ID and a print date and time indicated by the extracted print date and time information and displays the generated notification screen TG with the display unit 72.

As explained above, when receiving the scan data D3, the second in-hospital terminal 7 performs the operation in FIG. 7 when the received scan data D3 is the scan data D3 of the first print and performs the operation in FIG. 9 when the received scan data D3 is the scan data D3 of the second print. The terminal processor 710 of the second in-hospital terminal 7 determines whether to perform the operation in FIG. 7 or the operation in FIG. 9 according to a type of a scan image indicated by the scan data D3 and a type of information recorded in a code included in the scan image indicated by the scan data D3.

### 1-6. Configuration and Effects

As explained above, the information management system 1000 includes the inquiry management system 1 that manages the inquiry answer information J1. The information management system 1000 includes the print system 2 that causes the printing apparatus 5 to print the inquiry answer information J1 acquired from the inquiry management system 1. The information management system 1000 includes the second in-hospital terminal 7 that acquires the inquiry answer information J1 from the scan data D3 of the first print on which the inquiry answer information J1 is printed and stores the acquired inquiry answer information J1 in the database 80 on the isolated network NW3 isolated from the external network NW1 to which the inquiry management system 1 and the print system 2 are connected. The print system 2 causes the printing apparatus 5 to print the print history PH of the inquiry answer information J1. The second in-hospital terminal 7 acquires the print history PH of the inquiry answer information J1 from the scan data D3 of the second print on which the print history PH of the inquiry answer information J1 is printed and notifies storage omission of the inquiry answer information J1 in the database 80 based on the acquired print history PH of the inquiry answer information J1 and the scan history of the first print.

Accordingly, when storage omission of the inquiry answer information J1 occurs in the database 80 because of a loss of the first print, double feeding occurring in the scanner 6, or the like, the medical worker HC can grasp that the storage leakage of the inquiry answer information J1 to the database 80 has occurred. Thus, the medical worker HC instructs reprinting from the first in-hospital terminal 4 and causes the database 80 to store the inquiry answer information J1 by scanning a reprinted print. In this way, the medical worker HC can cope with the storage omission of the inquiry answer information J1 in the database 80.

The second in-hospital terminal 7 notifies storage omission in the database 80 about the inquiry answer information J1 included in the acquired print history PH of the inquiry answer information J1 and not included in a scan history of the first print, to the database 80.

Accordingly, when storage omission of the inquiry answer information J1 has occurred in the database 80, the medical worker HC can grasp about which inquiry answer information J1 the storage omission has occurred. Thus, the medical worker HC can accurately cope with the storage omission of the inquiry answer information J1 in the database 80.

The printing apparatus 5 generates a first print on which the inquiry answer information J1 is printed in a code format. The second in-hospital terminal 7 acquires the inquiry answer information J1 from the first code CD1 included in the scanned image indicated by the scan data D3 of the first print.

Accordingly, since the inquiry answer information J1 can be acquired without performing character recognition on a printing surface of the first print, it is possible to reduce a processing load at the time when the inquiry answer information J1 is stored in the database 80.

The second in-hospital terminal 7 urges reprinting of the inquiry answer information J1, storage of which in the database 80 is omitted and notifies the job ID of the first print on which the inquiry answer information J1, storage of which in the database 80 is omitted, is printed.

Accordingly, by urging the reprinting, it is possible to increase the motivation of the medical worker HC who is about to cope with the storage omission. Since the job ID is notified, the medical worker HC can easily grasp which first print is a first print requiring reprinting. Thus, it is possible to increase the possibility that the storage omission of the inquiry answer information J1 in the database 80 is accurately eliminated.

The second in-hospital terminal 7 further notifies a print date and time of the first print on which the inquiry answer information J1, storage of which in the database 80 is omitted, is printed.

Accordingly, since the medical worker HC can easily grasp on a first print at which date and time the reprinting is necessary, it is possible to further increase the possibility that the storage omission of the inquiry answer information J1 in the database 80 is accurately eliminated.

The information management system 1000 includes a reception unit 114 that receives designation of print time for printing the print history PH of the inquiry answer information J1. The print system 2 causes the printing apparatus 5 to print the print history PH of the inquiry answer information J1 based on the print time received by the reception unit 114.

Accordingly, since the printing apparatus 5 can generate the second print at a determined time, it is possible to prevent the medical worker HC from forgetting to scan the second print. Thus, when storage omission of the inquiry answer information J1 has occurred in the database 80, it is possible to increase the possibility that the medical worker HC can grasp that the storage omission of the inquiry answer information J1 has occurred in the database 80. Thus, it is possible to increase the possibility that the storage omission of the inquiry answer information J1 in the database 80 is eliminated.

The print system 2 causes the printing apparatus 5 to print the print history PH of the inquiry answer information J1 printed by the printing apparatus 5 after the second print generated last time.

Accordingly, by narrowing down the print history PH printed on the second print to the print history PH of the inquiry answer information J1 printed by the printing apparatus 5 after the previously generated second print, it is possible to reduce a processing amount for the print history PH in processing relating to storage omission of the inquiry answer information J1. Thus, the second in-hospital terminal 7 can quickly notify the storage omission of the inquiry answer information J1 in the database 80.

The second in-hospital terminal 7 includes the terminal control unit 70 that acquires the inquiry answer information J1 from the scan data D3 of the first print on which the inquiry answer information J1 is printed and stores the acquired inquiry answer information J1 in the database 80 on the isolated network NW3. The terminal control unit 70 acquires the print history PH of the inquiry answer information J1 from the scan data D3 of the second print on which the print history PH of the inquiry answer information J1 is printed and notifies storage omission of the inquiry answer information J1 in the database 80 based on the acquired print history PH of the inquiry answer information J1 and the scan history of the first print.

Accordingly, the same effects as the effects of the information management system 1000 explained above are achieved.

The scan APP 721 causes the terminal processor 710 of the second in-hospital terminal 7 to acquire the inquiry answer information J1 from the scan data D3 of the first print. The scan APP 721 causes the terminal processor 710 to store the acquired inquiry answer information J1 in the database 80 on the isolated network NW3. The scan APP 721 causes the terminal processor 710 to acquire the print history PH of the inquiry answer information J1 from the scan data D3 of the second print on which the print history PH of the inquiry answer information J1 is printed. The scan APP 721 causes the terminal processor 710 to notify storage omission of the inquiry answer information J1 in the database 80 based on the acquired print history of the inquiry answer information J1 and the scan history of the first print.

Accordingly, the same effects as the effects of the information management system 1000 explained above are achieved.

The print server 20 includes the print server control unit 200 that causes the printing apparatus 5 to generate the first print on which the inquiry answer information J1 is printed and causes the printing apparatus 5 to generate the second print on which the print history PH of the inquiry answer information J1 is printed. The first print is a print that is a target of the scan data D3 from which the inquiry answer information J1 is acquired by the second in-hospital terminal 7 that stores the inquiry answer information J1 in the database 80 on the isolated network NW3 isolated from the external network NW1. The second print is a print on which the print history PH of the inquiry answer information J1 compared with the scan history of the first print is printed when storage omission of the inquiry answer information J1 in the database 80 is notified.

Accordingly, the same effects as the effects of the information management system 1000 explained above are achieved.

### 2. Second Embodiment

### 2-1. Configuration of a Server

Subsequently, the second embodiment is explained.

When the second embodiment and the first embodiment are compared, the second embodiment is different from the first embodiment in configurations of the inquiry management server 10 and the print server 20.

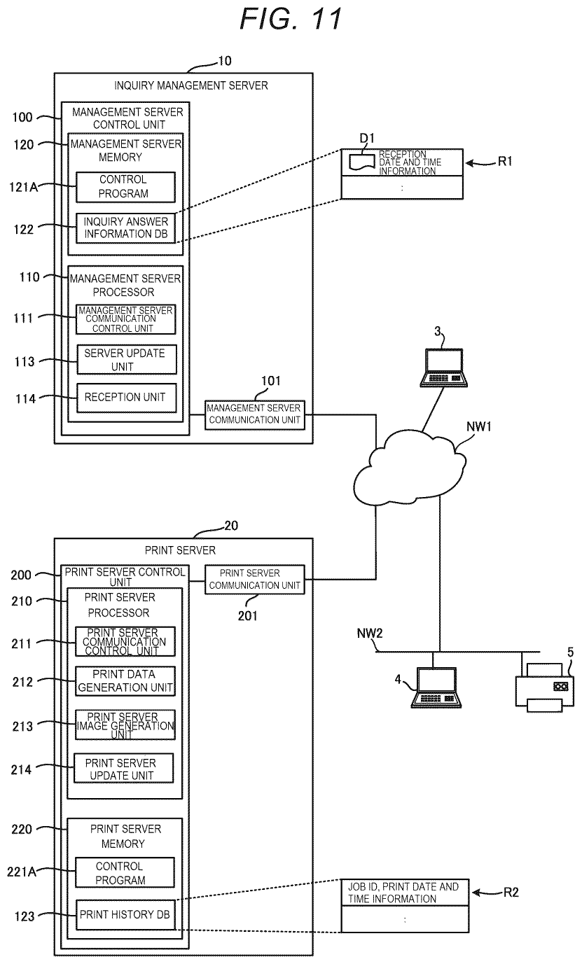

FIG. 11 is a diagram illustrating the configurations of the inquiry management server 10 and the print server 20 in the second embodiment.

In the explanation of the configurations of the inquiry management server 10 and the print server 20 in the second embodiment, differences from the first embodiment are explained.

In the second embodiment, the management server memory 120 does not store the print history DB 123. In the second embodiment, the management server memory 120 stores a control program 121A instead of the control program 121. The control program 121A is a program for causing the management server processor 110 to function as the management server communication control unit 111, the server update unit 113, and the reception unit 114. That is, in the second embodiment, the management server processor 110 does not function as the image generation unit 112.

In the second embodiment, the print server memory 220 stores the print history DB 123. In the second embodiment, the print server memory 220 stores a control program 221A instead of the control program 221. The control program 221A is a program for causing the print server processor 210 to function as the print server communication control unit 211, the print data generation unit 212, a print server image generation unit 213, and a print server update unit 214. That is, in the second embodiment, the print server processor 210 further functions as the print server image generation unit 213 and the print server update unit 214.

In the second embodiment, the print server communication control unit 211 receives the inquiry answer information data D1 from the inquiry management system 1. Like the image generation unit 112, the print server image generation unit 213 generates the first print image PG1 based on the inquiry answer information data D1 received by the print server communication control unit 211. After generating the first print image PG1, the print server image generation unit 213 outputs image data of the generated first print image PG1 to the print data generation unit 212. When receiving the image data of the first print image PG1, the print data generation unit 212 generates, based on the received image data of the first print image PG1, print data for printing the first print image PG1. The print data D2 generated by the print data generation unit 212 is transmitted to the printing apparatus 5.

Like the image generation unit 112, the print server image generation unit 213 generates the second print image PG2 referring to the print history DB 123. After generating the second print image PG2, the print server image generation unit 213 outputs image data of the generated second print image PG2 to the print data generation unit 212. When receiving the image data of the second print image PG2, the print data generation unit 212 converts the received image data of the second print image PG2 into print data. The print data D2 generated by the print data generation unit 212 is transmitted to the printing apparatus 5.

The print server update unit 214 updates the print history DB 123. The print server update unit 214 generates the record R2 when the print server image generation unit 213 generates the first print image PG1 and stores the generated record R2 in the print history DB 123. When generating the record R2, the print server update unit 214 acquires a job ID generated at the time of generating the first print image PG1 from the print server image generation unit 213. Then, the print server update unit 214 generates the record R2 including the acquired job ID and print date and time information. In the present embodiment, the print date and time indicated by the print date information included in the record R2 may be a date and time when the image data of the first print image PG1 was generated, may be a date and time when the print system 2 caused the printing apparatus 5 to print the first print image PG1, or a date and time when the printing apparatus 5 completed the printing of the first print image PG1.

### 2-2. Operation Until the Inquiry Answer Information is Stored

Subsequently, operations of the units of the information management system 1000 in the present embodiment are explained.

When the second embodiment and the first embodiment are compared, the second embodiment is different from the first embodiment in operations of the inquiry management server 10 and the print server 20 until the printing apparatus 5 generates the first print.

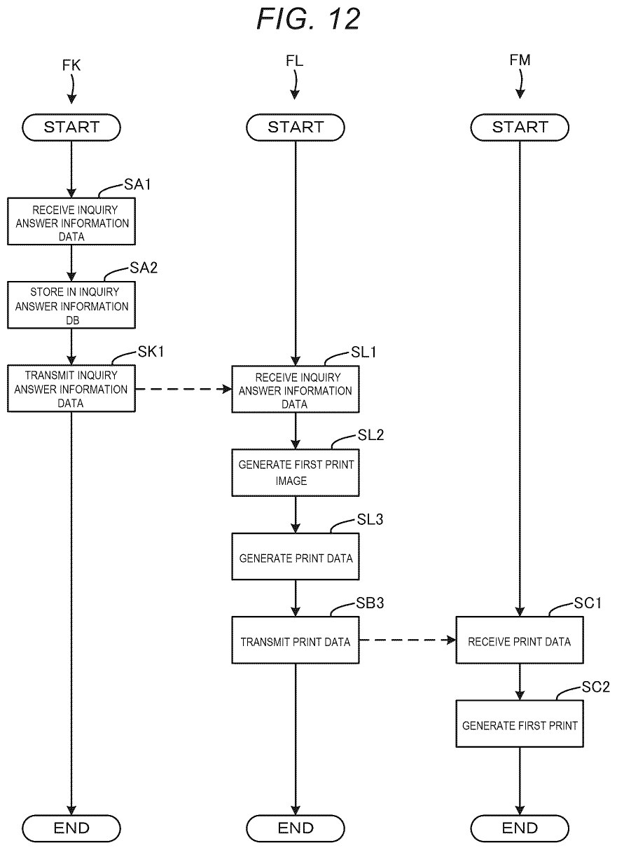

FIG. 12 is a flowchart illustrating the operations of the units of the information management system 1000. In FIG. 12, a flowchart FK illustrates an operation of the inquiry management system 1, a flowchart FL illustrates an operation of the print system 2, and a flowchart FM illustrates an operation of the printing apparatus 5.

In the flowcharts illustrated in FIG. 12, the same steps as the steps in the flowcharts illustrated in FIG. 6 are denoted by the same step numbers and detailed explanation of the steps is omitted.

As illustrated in the flowchart FK, the management server communication control unit 111 transmits the inquiry answer information data D1 received in step SA1 to the print system 2 (step SK1).

As illustrated in the flowchart FL, the print server communication control unit 211 receives the inquiry answer information data D1 from the inquiry management system 1 (step SL1).

Subsequently, the print server image generation unit 213 generates the first print image PG1 based on the inquiry answer information data D1 received in step SL1 (step SL2).

Subsequently, the print data generation unit 212 generates the print data D2 for printing the first print image PG1 generated in step SL2 (step SL3).

### 2-3. Operation Relating to Storage Omission of the Inquiry Answer Information

The second embodiment is different from the first embodiment in operations of the inquiry management server 10 and the print server 20 until the printing apparatus 5 generates the second print.

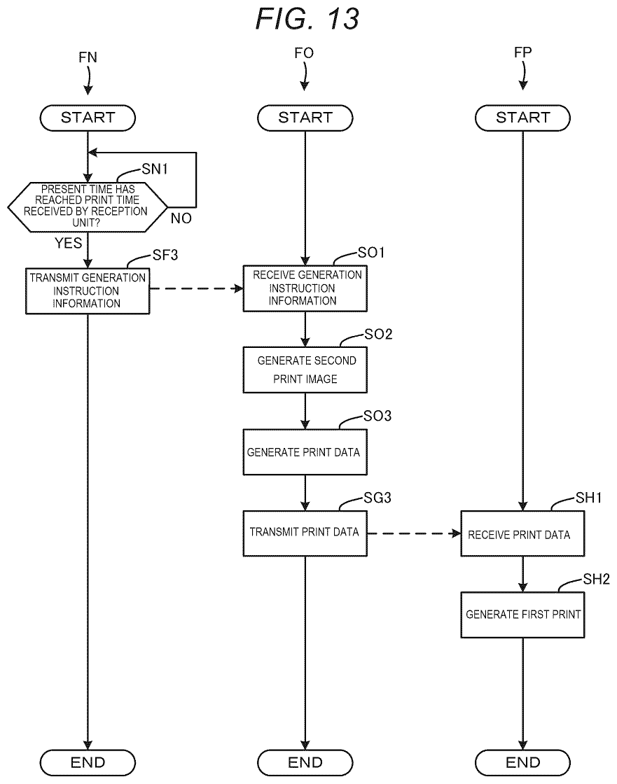

FIG. 13 is a flowchart illustrating operations of the units of the information management system 1000. In FIG. 13, a flowchart FN illustrates an operation of the inquiry management system 1, a flowchart FO illustrates an operation of the print system 2, and a flowchart FP illustrates an operation of the printing apparatus 5.

In the flowcharts illustrated in FIG. 13, the same steps as the steps in the flowcharts illustrated in FIG. 8 are denoted by the same step numbers and detailed explanation of the steps is omitted.

The management server communication control unit 111 determines whether the present time has reached a time received by the reception unit 114 (step SN1). When determining that the present time has not reached the time received by the reception unit 114 (step SN1: NO), the management server communication control unit 111 performs the determination in step SN1 again.

When determining that the present time has reached the time received by the reception unit 114 (step SN1: YES), the management server communication control unit 111 transmits generation instruction information indicating a generation instruction for the second print to the print system 2 (step SN2).

As illustrated in the flowchart FG, the print server communication control unit 211 receives the generation instruction information from the inquiry management system 1 (step SO1).

Subsequently, the print server image generation unit 213 generates the second print image PG2 (step SO2).

Subsequently, the print data generation unit 212 generates print data D2 for printing the second print image PG2 generated in step SO2 (step S03).

### 2-4. Effects

According to the present embodiment, the same effects as the effects of the first embodiment are achieved.

### 3. Other Embodiments

The embodiments explained above indicate an aspect and can be optionally modified and applied.

In the embodiments explained above, the management server processor 110 functions as the reception unit 114. However, in another embodiment, the print server processor 210 may function as the reception unit 114. In the other embodiment, the first in-hospital terminal 4 communicates with the print system 2.

In the embodiments explained above, the storage omission in the database 80 is notified by the display. However, a notification form of the storage omission in the database 80 is not limited to the display and may be another form such as voice output or transmission of an e-mail.

In the embodiments explained above, the case in which the number of the scanners 6 connected to the isolated network NW3 and the number of the printer apparatus 5 connected to the external connection network NW2 are one is exemplified. However, the number of the scanners 6 connected to the isolated network NW3 and the number of the printing apparatus 5 connected to the external connection network NW2 are not limited to one and may be plural.

In the embodiments explained above, the information management system 1000 is applied to the hospital H. However, a target to which the information management system 1000 is applied is not limited to the hospital H. The information management system 1000 can also be applied to a facility or an entity that manages personal information, confidential information, and the like with an isolated network. In this case, the “management system” manages “management information” such as personal information and confidential information, and the “management device” is connected to the isolated network and stores the “management information” such as personal information and confidential information in the “database” on the isolated network. For example, targets other than the hospital H to which the information management system 1000 is applied are government offices and police.

In the embodiments explained above, the job ID and the inquiry answer information J1 are acquired from the first code CD1 by printing the first code CD1. In another embodiment, the first code CD1 may not be printed, the job ID and the inquiry answer information J1 may be printed, and the job ID and the inquiry answer information J1 may be acquired from a scanned image by optical character recognition (OCR).

In the embodiments explained above, the print history PH is acquired from the second code CD2 by printing the second code CD2. In another embodiment, the print history PH may be printed without printing the second code CD2 and the job ID and the print date and time information may be acquired from the scanned image by the OCR.

In another embodiment, the functions of the management server processor 110, the print server processor 210, and the terminal processor 710 may be implemented by a plurality of processors or a semiconductor chip.

The units of the inquiry management server 10, the print server 20, and the second in-hospital terminal 7 illustrated in FIGS. 2, 5, and 11 are examples and are not particularly limited. Pieces of hardware individually corresponding to the units do not always need to be installed, and, naturally, one processor can be configured to execute a program to implement the functions of the units. A part of functions implemented by software may be implemented by hardware or a part of functions implemented by hardware may be implemented by software. Besides, specific detailed configurations of the other units of the inquiry management server 10, the print server 20, and the second in-hospital terminal 7 can be optionally changed.

The step units of the operations illustrated in FIGS. 6, 7, 8, 9, 11, 12, and 13 are divided according to main processing contents in order to facilitate understanding of the operations of the units of the information management system 1000 and are not limited by a way of division and names of processing units. According to the processing contents, the operations may be divided into more step units. The operations may also be divided such that one step unit includes more processing. The order of the steps may be changed as appropriate.

### 4. Summary of the Present Disclosure

A summary of the present disclosure is appended below.

### Appendix 1

An information management system including: a management system configured to manage management information; a print system configured to cause a printing apparatus to print the management information acquired from the management system; and a management device configured to acquire the management information from scan data of a first print on which the management information is printed and store the acquired management information in a database on an isolated network isolated from an external network, wherein the print system causes the printing apparatus to print a print history of the management information, and the management device acquires the print history of the management information from scan data of a second print on which a print history of the management information is printed and notifies storage omission of the management information in the database based on the acquired print history of the management information and a scan history of the first print.

According to this configuration, when the storage omission of the management information has occurred in the database on the isolated network because of a loss of the print on which the management information is printed or double feeding occurring in a scanner, a user of the management device can grasp that the storage omission of the management information has occurred in the database on the isolated network. Thus, the user of the management device performs reprinting and causes the database to store the management information by scanning a reprinted print. In this way, the user can cope with the storage omission of the management information in the database.

### Appendix 2

The information management system described in the appendix 1, wherein the management device notifies storage omission in the database about the management information included in the acquired print history of the management information and not included in the scan history.

According to this configuration, when the storage omission of the management information has occurred in the database on the isolated network, the user of the management device can grasp about which management information the storage leakage has occurred. Thus, the user of the management device can accurately cope with the storage omission of the management information in the database.

### Appendix 3

The information management system described in the appendix 1 or 2, wherein the printing apparatus generates the first print on which the management information is printed in a code format, and the management device acquires the management information from a code included in a scan image indicated by the scan data of the first print.

According to this configuration, the management information can be acquired without performing character recognition on a printing surface of the first print. Therefore, it is possible to reduce a processing load at the time when the management information is stored on the database on the isolated network.

### Appendix 4

The information management system described in any one of appendices 1 to 3, wherein the management device urges reprinting of the management information, storage of which in the database is omitted, and notifies the identification information of the first print on which the management information, storage of which in the database is omitted, is printed.

According to this configuration, by urging the reprinting, it is possible to increase the motivation of the user of the management device who is about to cope with the storage omission. Since the identification information is notified, the user of the management device can easily grasp which the first print requires the reprinting. Thus, it is possible to increase the possibility that the storage omission of the management information in the database is accurately eliminated.

### Appendix 5

The information management system described in the appendix 4, wherein the management device further notifies a print date and time of the first print on which the management information, storage of which in the database is omitted, is printed.

According to this configuration, the user of the management device can easily grasp the first print at which date and time the reprinting is necessary. Therefore, it is possible to further increase the possibility that the storage omission of the management information in the database is accurately eliminated.

### Appendix 6

The information management system described in any one of the appendices 1 to 5, further including a reception unit configured to receive designation of print time for printing the print history of the management information, wherein the print system causes the printing apparatus to print the print history of the management information based on the print time received by the reception unit.

According to this configuration, since the printing apparatus can generate the second print at a determined time, it is possible to prevent the user of the management device from forgetting to scan the second print. Thus, when storage omission of the management information has occurred in the database on the isolated network, it is possible to increase the possibility that the user of the management device can grasp that the storage omission of the management information has occurred in the database. Accordingly, it is possible to increase the possibility that the storage omission of the management information in the database is eliminated.

### Appendix 7

The information management system described in any one of appendices 1 to 6, wherein the print system causes the printing apparatus to print a print history of the management information printed by the printing apparatus after the second printing object generated last time.

According to this configuration, by narrowing down the print history printed on the second print to the print history of the management information printed by the printing apparatus after the second print generated last time, it is possible to reduce a processing amount for the print history in processing relating to the storage omission of the management information. Thus, the management device can quickly notify the storage omission of the management information in the database.

### Appendix 8

A management device connected to an isolated network isolated from an external network to which a management system that manages management information and a print system that causes a printing apparatus to print the management information acquired from the management system are connected, the management device including a management device control unit configured to acquire the management information from scan data of a first print on which the management information is printed and store the acquired management information in a database on the isolated network, wherein the management device control unit acquires a print history of the management information from scan data of a print that the print system caused the printing apparatus to print, the print being a second print on which the print history of the management information is printed, and notifies storage omission of the management information in the database based on the acquired print history of the management information and a scan history of the first print.

According to this configuration, the same effects as the effects of the information management system described in the appendix 1 are achieved.

### Appendix 9

A non-transitory computer-readable storage medium storing a program, the program causing a processor of a management device connected to an isolated network separated from an external network, to which a management system that manages management information and a print system that causes a printing apparatus to print the management information acquired from the management system are connected, to: acquire the management information from scan data of a first print on which the management information is printed; store the acquired management information in a database on the isolated network; acquire a print history of the management information from scan data of a print that the print system caused the printing apparatus to print, the print being a second print on which the print history of the management information is printed; and notify storage omission of the management information in the database based on the acquired print history of the management information and a scan history of the first print.

According to this configuration, the same effects as the effects of the information management system described in the appendix 1 are achieved.

### Appendix 10

A server connected to, via an external network, a management system that manages management information, the server causing a printing apparatus to print the management information acquired from the management system, the server including a server control unit configured to cause the printing apparatus to generate a first print on which the management information is printed and cause the printing apparatus to generate a second print on which a print history of the management information is printed, wherein the first print is a print that is a target of scan data from which a management device, which stores the management information in a database on an isolated network isolated from the external network, acquires the management information, and the second print is a print on which a print history of the management information to be compared with a scan history of the first print is printed when storage omission of the management information in the database is notified.

According to this configuration, the same effects as the effects of the information management system described in the appendix 1 are achieved.

What is claimed is:

1. An information management system comprising:
a management system configured to manage management information; a print system configured to cause a printing apparatus to print the management information acquired from the management system; and
a management device configured to acquire the management information from scan data of a first print on which the management information is printed and store the acquired management information in a database on an isolated network isolated from an external network, wherein
the print system causes the printing apparatus to print a print history of the management information, and the management device acquires the print history of the management information from scan data of a second print on which a print history of the management information is printed and notifies storage omission of the management information in the database based on the acquired print history of the management information and a scan history of the first print.

2. The information management system according to claim 1, wherein the management device notifies storage omission in the database about the management information included in the acquired print history of the management information and not included in the scan history.

3. The information management system according to claim 1, wherein the printing apparatus generates the first print on which the management information is printed in a code format, and the management device acquires the management information from a code included in a scan image indicated by the scan data of the first print.

4. The information management system according to claim 1, wherein the management device urges reprinting of the management information, storage of which in the database is omitted, and notifies the identification information of the first print on which the management information, storage of which in the database is omitted, is printed.

5. The information management system according to claim 4, wherein the management device further notifies a print date and time of the first print on which the management information, storage of which in the database is omitted, is printed.

6. The information management system according to claim 1, further comprising a reception unit configured to receive designation of print time for printing the print history of the management information, wherein the print system causes the printing apparatus to print the print history of the management information based on the print time received by the reception unit.

7. The information management system according to claim 1, wherein the print system causes the printing apparatus to print a print history of the management information printed by the printing apparatus after the second printing object generated last time.

8. A management device connected to an isolated network isolated from an external network to which a management system that manages management information and a print system that causes a printing apparatus to print the management information acquired from the management system are connected,
the management device comprising a management device control unit configured to acquire the management information from scan data of a first print on which the management information is printed and store the acquired management information in a database on the isolated network, wherein
the management device control unit acquires a print history of the management information from scan data of a print that the print system caused the printing apparatus to print, the print being a second print on which the print history of the management information is printed, and notifies storage omission of the management information in the database based on the acquired print history of the management information and a scan history of the first print.

9. A non-transitory computer-readable storage medium storing a program, the program causing a processor of a management device connected to an isolated network separated from an external network, to which a management system that manages management information and a print system that causes a printing apparatus to print the management information acquired from the management system are connected, to:
acquire the management information from scan data of a first print on which the management information is printed;
store the acquired management information in a database on the isolated network;
acquire a print history of the management information from scan data of a print that the print system caused the printing apparatus to print, the print being a second print on which the print history of the management information is printed; and
notify storage omission of the management information in the database based on the acquired print history of the management information and a scan history of the first print.
